(19) United States
(12) Patent Application Publication
(10) Pub. No.: US 2020/0312458 A1
(43) Pub. Date: Oct. 1, 2020

(54) SYSTEM AND METHOD FOR ENCRYPTED GENUINE GATHERING

(71) Applicants: Jennie Luna, Allendale, NJ (US); Vivek Oberol, Chester, NY (US)

(72) Inventors: Jennie Luna, Allendale, NJ (US); Vivek Oberol, Chester, NY (US)

(21) Appl. No.: 16/834,939

(22) Filed: Mar. 30, 2020

Related U.S. Application Data
(60) Provisional application No. 62/826,858, filed on Mar. 29, 2019.
(60) Provisional application No. 62/934,463, filed on Nov. 12, 2019.

(51) Int. Cl. G16H 50/20; G16H 80/00; G16H 10/60; G06Q 50/18

(52) U.S. Cl. CPC G16H 50/20; G06Q 50/18; G16H 10/60; G16H 80/00

(57) ABSTRACT

Disclosed herein is a system for receiving and distributing a patient's medical, health, and lifestyle history in a single repository. The system will also receive and provide, in an online marketplace, a broad range of services and service providers relevant to a patient's medical status as well as general lifestyle services related to the overall health and wellness of a patient. The system's single repository will keep a user's health data organized, decrease cost from unnecessary testing, personalize patient care and effectively track high risk patients. By creating a single repository, the system empowers the individual to be the owner of all their health information in a clear and updated, genuine view.

### CROSS-REFERENCE TO RELATED PATENT APPLICATIONS

The present application claims priority to U.S. Provisional Patent Application Serial Nos. 62/826,585, filed Mar. 29, 2019 and 62/934,463, filed Nov. 12, 2019.

### BACKGROUND OF THE INVENTION

### 1. Field of the Invention

Embodiments of the invention relate to a system and method directed to a comprehensive and proactive approach to medical care and records.

### 2. Description of Related Art

The health expenditure in the United States as per the CDC is $3.3 trillion with a GDP of 17.9%. Our health care costs are higher and not necessarily showing better outcomes when compared to other countries. There are several culprits, but at the root of the problem is a massively disorganized system and a lack of communication.

There are several factors that drive the ever-increasing costs of healthcare. Lack of personalized medicine that results in hospital admissions. There are high-risk patients that are not cared for properly and become “frequent flyers” in the hospital. Many of which use the Emergency Room (ER) as a primary care service. Many patients feel they are “just another number.”

A disorganized and miscommunicated system has resulted in inefficient care of the patient despite medical advances and technology. Not having convenient ownership and a comprehensive understanding of their medical care and records inhibits the patient to proactively be part of their health and subsequent prevention of disease. Doctors do not share a collective patient record, so they repeat tests and cannot effectively manage the patient. This results in wasteful spending. In addition, the doctors are only seeing a fraction of the patient's complete medical care. In the absence of a patient's complete medical report, each provider is focused only on their specific area without taking the initiative to guide the patient and family for palliative care. As a result, chronic diseases like diabetes, obesity and heart diseases, which are related to lifestyle, are not addressed appropriately and prevention is not a priority in the current healthcare system.

Cost for healthcare is ever-increasing and not predictable. For example, cost variation for services in doctor offices, hospitals, specialist is unknown and usually not revealed until the patient is billed. Prescription drugs are outrageously priced, and every year pharmaceutical companies increase their prices more. Since patients don't know how to shop for cheaper options, they end up settling for what's offered.

Many patients do not seek medical treatment, skip medications, and/or don't fill their prescriptions when their deductibles are high or when Medicare patients reach the “Donut Hole” which happens when all of their coverage for the year was used. As a result, there is worsening in the health condition of the individual and possible Emergency Room visits.

Healthcare providers are practicing medicine with the same methods as they did decades ago when the population and health problems were very different. But recent advances with Electronic Medical Records (EMR) has not improved patient care. Ineffective practices have continued to pile on, and no one is taking full leadership of the system. Therefore a need exists for a platform to address the current state of our healthcare system.

### SUMMARY OF THE INVENTION

The embodiments of the invention are provide a number of advantages to its user. The platform guides providers with the best care for patients since they will have a complete view of their medical history. The platform will help doctors track cost, market analysis, and find areas for improvement. The platform structures health information in a clear, comprehensive format for each provider. Rare genetic diseases are quickly identified for immediate entry into a clinical trial by connecting with other facilities posting clinical trial through the platform.

The platform allows doctors to reach a broader market, and patients have easier access to health services matched to their needs. Accurate documentation will be convenient and more effective, allowing insurance companies to see a clear clinical view of the patient. Reviews will be left by individuals who are real consumers of a plurality of service providers, unlike the current review system that is not completely honest. Medical providers can post the insurances they accept, making it easier for patients' to pick in-network services.

Patients will have convenient ownership and a comprehensive understanding of their medical care and records. This will allow patient users and their caregivers the opportunity to proactively be part of their health. The platform will provide a medical service provider a patient's complete medical history. This will save time for both the patient and the medical provider and improve the quality of care.

The platform will help reduce wasteful spending as the medical service provider will have access to all the previously performed laboratory tests and results. This will help bring down the cost of end of life care as medical service providers will be able to access other medical providers' notes and guide the patient and family for palliative care. Reduced medication errors and repeated tests will eliminate wasteful spending. As a large data repository, in which artificial intelligence can be used, will improve both the patient's quality of life and treatment. The platform will better track and offer a clear picture of the individual's overall health.

The platform will offer more convenient doctor visits for the patient given their entire health history is available in one location. In this regard, the platform is a tool for proactive and personal care. Essentially the individual is the owner of all their health information in a clear and updated, genuine view. This will keep the patient user's health data organized, decrease cost from unnecessary testing, personalize patient care and effectively track high risk patients.

### DETAILED DESCRIPTION OF EMBODIMENTS

While the technology is illustrated and described in a preferred embodiment, the platform may be produced and described in many different configurations, forms and various methods, without deviating from the scope of the present invention. The preferred embodiment of the invention is described in detail, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the technology described herein.

Embodiments of the present invention are direct to methods, systems, and computer storage media for receiving and distributing a patient's medical, health, and lifestyle history in a single repository. The methods, systems, and computer storage media will also receive and provide, in an online marketplace, a broad range of services and service providers relevant to a patient's medical status as well as general lifestyle services related an overall health and wellness of a patient.

In a preferred embodiment of the present invention, the platform provides a single repository for the records of a patient's comprehensive health history. The platform is a cloud-based platform. The electronic medical record of the patient is maintained in the platform by a health data module in the form of a health capsule. The data maintained in the health capsule of the patient is collected by the health data module in several ways and encompasses a comprehensive medical snapshot of the patient's overall health.

In one embodiment, the health data module automatically collects patient information from institutions such as Clearinghouse, Commonwell, and/or Carequality. The personal health information associated with the platform may be created at any time during the lifetime of the patient. The data collected by the health data module, relative to the medical history of the patient, can only be received from a medical service provider, hospital, or similar institution. In this aspect, the health data module is not authorized to collect medical history data from the patient or any unauthorized group or individual.

Alternatively, the patient can request the health data module provide a medical service provider a continuity of care document. The continuity of care document may be provided electronically. In an alternative embodiment, the health data module automatically sends the continuity of care document to a service provider and automatically updates the patient's personal health information. In this regard, the medical service provider's access to the patient's health capsule is limited to the continuity of care document provided by the platform.

In a preferred embodiment, the health capsule associated with the platform is created at birth. In this preferred embodiment, the health data module collects medical history data from in utero until death of the patient. An advantage of this embodiment is the ability of the health data module to receive and maintain a child's medical records, vaccination records, and monitor growth and development. A child's parent can therefore have in a single repository all of their child's EMR's until the child is over the age of 18 or emancipated. In this aspect, family members may connect their respective health capsule through the platform.

In a preferred embodiment, the patient determines through the platform who is authorized to access their health capsule. The patient may set various parameters related to authorization by setting expiration timeframes and termination dates.

A medical service provider who has been authorized to access the platform and health capsule of the patient may provide laboratory results and imaging reports for collection by the health data module to update the health capsule of the patient. The health capsule will have age, gender, vitals, medications, family history, surgeries, all medical notes, hospitalizations, health trend, and insurance information. The health capsule of the platform may be prepopulated with diagnosis codes, which paints a clear picture of the patient for the insurance company. Advantageously for the medical service provider is the ability to record findings, assessment, and plan and avoid writing redundant notes.

In some embodiments, the platform will send an alert to a medical services provider when an individual has been admitted to the hospital. An advantage of this embodiment, is increased focus on those patients requiring more attention. As a result, there will be a decrease in cost and hospital admissions. Further, when a patient is admitted to the hospital, the treating medical providers will be able to pull data from the health capsule. The medical provider will therefore have access to a patient's comprehensive medical and health history. Upon discharge from the hospital, the health data module is configured to capture the data created during their admission including hospital admission notes, medications provided, tests and laboratory results as well as any associated imaging performed.

In another preferred embodiment of the invention, the platform includes a communication module to facilitate remote video conferencing connections with services providers, which includes but is not limited to a medical service provider, coaches, tutors, language specialists, doctors, providers, fitness mentors, wellness guides, insurance companies, and the like. The communication module connects the patient with those service providers that have joined and/or created an account in the platform.

The platform collects the information of the various service providers by way of a service provider module. The service provider module receives a request to join the platform and collects the service provider's information including but not limited to their area of expertise, prices, schedule, and the like. For example, member pharmacies may be linked to the patient's health capsule and will identify those medications covered by insurance. The service provider can control what information is collected by the service provider module and what is viewable by individuals and patients through the platform. While a patient user of the platform may be able to view a service providers offerings, the service provider will only be permitted to see the information associated with a patient user unless and until they have been provided authorization by the user patient. The platform is advantageous to the service provider as providing an online marketplace for their service offerings. In this aspect, services rendered to users of the platform will be made safely and conveniently through the platform. Transactions will be conducted conveniently through the health capsule. A percentage will be assessed for use of the platform. While there is no fee assessed for a service provider to open an account, additional features selected by the service provider may include a fee such as modules to track cost, market analysis, identify areas for improvement, and paid advertising. In addition, any marketing and/or advertising by the service providers holding an account are created by those service providers. The use of the platform by a patient user is at no charge.

The communication module may receive data from a patient regarding their current health status. The communication module can connect the individual with a telehealth medical provider who can triage the patient to either local urgent care or emergency room if needed. The medical provider can report to the accepting provider and give a full report of the individual's complaints and reason for referral. An advantage of this aspect of the invention is the decrease in automatic 911 calls that result in unnecessary emergency room or urgent care visits.

Another preferred embodiment of the invention is the ability to connect a patient platform user with their current medical service providers. The patient user may request the platform initiate an electronic request to a particular medical service provider to join or create an account in the platform. Once the medical service provider has joined the platform, that particular medical service provider is able to view and download information from the patient's health capsule to which the medical service provider has been granted access. It is to be understood that the transfer of any personal health information through the platform and the health capsule is compliant with the Health Insurance Portability and Accountability Act (HIPAA). Access is granted to the health capsule by means known to one of ordinary skill in the art including, but not limited to, via a code populated by the health data module, which may be provided to a medical services provider, by biometrics, i.e., fingerprint scanner, a drop of blood, iris scanner, or a by a chip card.

A platform service provider can automatically add appointments, which can be tracked through the platform by the patient user. As a non-limiting example, a medical service provider can schedule a future follow-up medical appointment, which is added automatically by the health data module. The health data module, in this aspect, is configured to receive scheduling information, which is maintained in a patient calendar in the platform. The platform may be configured by the patient user to merge schedules with their children and/or elderly parents. The platform is configurable to remind patients about appointments and tasks to be completed related to lifestyle, health and wellness. It is also the intent of this invention to allow merger of the platform calendaring embodiment with any of a user's calendaring systems so as to have one single location for all calendaring.

The health data module is configured to pull data from the patient's insurance company thereby making services more transparent and easier for the patient to determine what is covered. In an additional embodiment, the platform is enabled to pull data from a plurality of insurance companies by way of the service provider module to give recommendations for insurance if a patient user is not covered. The data pulled from a plurality of insurance companies will be relevant to a particular patient user based on their location, income, health problems, familial makeup, etc. Because the health data module is configured to pull data from the patient's insurance company, the yearly insurance spending will be tracked.

In another embodiment of the invention, the platform includes a lifestyle module. The lifestyle module is configured to receive data from social media use, and overall health and wellness of the patient user. For example, the lifestyle module will connect with a plurality of wellness and fitness applications to track health and wellness of the patient user. The lifestyle module will track sleep, stress management, nutrition and physical activity by receiving data from other health devices (smart watches, cellphones, health trackers) as well as membership and attendance information associated with wellness services. When a patient user has increasing risk factors such as high body mass index, diabetes, or uncontrolled blood pressure, the platform may be configured to provide an alert to a selected medical service provider and the patient user. The patient can set health and wellness goals through the lifestyle module. The data received by the lifestyle module can be received by the health data module to updated the health capsule and provide a total health and wellness picture of the patient for any treating medical service provider. The platform tracks the wellbeing of the individual by learning their health trends, patterns, and behaviors. The lifestyle module may be accessible by user selected insurance companies to lower premiums based on the user's health status.

In some embodiments, the user can create and/or join community sites organized for the purpose of connecting platform members with similar interests, struggles, hobbies, goals, etc.

In another embodiment of the invention, the health data module is configured to provide legal documents relative to the patient's end of life instructions. For example, a primary medical provider may prompt an inquiry of the patient to determine any end of life instructions. The health data module receives the instruction in the form of a do not resuscitate instruction, a power of attorney instruction, organ donation, etc. Access to the legal documentation associated with a patient's end of life instruction may be restricted by the patient user and only accessible to those provided authorization to access the instructions. Access may be granted by way of an authentication code, token, password, or the like.

Because the platform collects data over the life of a patient user, from birth to death, valuable insight relevant to cancer research, chronic diseases, genetic disorders, gender, race, etc. are obtainable. In this aspect, the patient user can select and consent to share this information after their passing. Similarly, the patient user can create a lifelong digital story for families to remember them by whereby the patient user highlights significant events maintained in the platform repository. For example, a patient user may highlight a child's birth, first walk, graduation, etc. and attach videos and/or photos to be uploaded with the highlighted event and maintained in the repository.

In another embodiment of the invention, the health capsule may include links to educational information related to specific health related issues, medications, or lab results of the patient. In this aspect, the health data module identifies publicly available resources and is configured to provide a link to the source for further education of the patient. Moreover, the health data module may be configured to provide an overall summary of a patient's medical history based on a comprehensive compilation of data associated not only with the health capsule but also incorporating lifestyle data. The summary data may include trends related to lab values, vitals, and any health related data to map the data over a user selected period of time.

The health capsule provides a comprehensive view of a patient's overall health and wellbeing. Consistent with this approach, the health data module is configured to identify products and/or medications presenting a potential adverse reaction in the patient. For example, an allergy to a particular chemical or food may be flagged and the patient user alerted. Conversely, the health capsule may provide recommendations of products and/or medications safe for the patient user.

Because the health capsule is designed to be an interactive tool for the patient, a certain level of trust is built between the patient user and the health capsule. In this regard, the patient can rely on their health capsule to obtain education on a medical condition, medication, tests, doctor credentials, reviews, insurance coverage, and connection with a doctor when ill. The patient can interact with their health capsule by voice command, email, text, or other electronic communication. The health capsule may be personalized to reflect a desired color, name, or design. The comprehensive health capsule functions as a single repository for gathering health data from a plurality of applications and sources to paint a clear picture of the user and his/her life patterns.

Those of skill in the art will appreciate that the various illustrative logical blocks, module, units, and steps described in connection with the embodiments disclosed herein can often be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends on the particular constraints imposed on the overall system. Skilled persons can implement the described functionality in varying ways for each particular system, but such implementation decisions should not be interpreted as causing a departure from the scope of the invention. In addition, the grouping of functions within a unit, module, block, or step is for ease of description. Specific functions or steps can be moved from one unit, module, block, or step without departing from the invention.

The system of the present invention may be generally described in the context of computer executable instructions including program modules executed by a computer. The system of the present invention may include one or more networked devices that include at least one processor, at least one memory device, and an operating system configured to perform executable instructions. In further embodiments, a networked device includes software applications comprising a health data module, a lifestyle module, a communications module, and a service provider module. The networked device may be a digital processing device and includes one or more hardware central processing units (CPU) that carry out the device's functions. In further embodiments, the networked device includes an operating system configured to perform executable instructions. The operating system is, for example, software, including programs and data, which manages the device's hardware and provides services for execution of applications. In some preferred embodiments, the operating system is provided by cloud computing.

The system of the present invention includes a data repository coupled to at least one server and in communication with one or more networked devices. The data repository is configured to store information and databases for use by a user and the one or more networked devices. The information and databases stored in the data repository is configured to be searchable for one or more items of information by the various software applications and modules of the present invention. The network described herein may include, without limitation, one or more local area networks, wide area networks, and may be a secure network associated with a healthcare facility.

The computing environment of the system is exemplary, and it is to be understood that the various components and system are scalable. In this respect, the description of the system is for clarity only and not meant to limit the scope of the embodiments in any way.

The above description of the disclosed embodiments, and that provided in the accompanying documents, is provided to enable any person skilled in the art to make or use the invention. Various modifications to these embodiments will be readily apparent to those skilled in the art, and the generic principles described herein, and in the accompanying documents, can be applied to other embodiments without departing from the spirit or scope of the invention. Thus, it is to be understood that the description and drawing presented herein, and presented in the accompanying documents, represent particular aspects and embodiments of the invention and are therefore representative examples of the subject matter that is broadly contemplated by the present invention. It is further understood that the scope of the present invention fully encompasses other embodiments that are, or may become, obvious to those skilled in the art and that the scope of the present invention is accordingly not limited by the descriptions presented herein, or by the descriptions presented in the accompanying documents.

We claim:

1. A system for providing a platform to create comprehensive health data associated with a plurality of users, comprising:
at least one user data repository to store: a database for storing personal health information of the user; a lifestyle database, wherein data tracking the health and wellness of the user is stored; a legal document database, wherein one or more documents governing the user's end-of-life instructions are stored; and
a server coupled to the database for storing personal health information, lifestyle database, and legal document database via a network, wherein the server is in communication with a plurality of applications using a software application;
wherein the software application comprises:
a health data module configured to receive personal health information of the user, insurance information, legal documents governing the user's end-of-life instruction, and wherein the health data module is configured to receive data from a lifestyle module configured to receive data from a plurality of wellness and fitness applications that track health and wellness of the user;
a communications module to connect the user with a service provider, wherein the service provider includes a medical service provider, coach, tutor, language specialist, fitness mentor, wellness guide, or insurance company;
a service provider module configured to receive and provide to the user a plurality of services offered by a plurality of service providers associated with an account in the platform; and
wherein the at least one user data repository provides a comprehensive picture of the overall health of the user derived from information received by the software application.

2. The system of claim 1, wherein the health data module automatically sends a continuity of care document to a user selected service provider and automatically updates the patient's personal health information in the database.

3. The system of claim 2, wherein the user determines who is authorized to access their user data repository and sets a plurality of parameters related to authorization by setting expiration timeframes and termination dates.

4. The system of claim 3, wherein access to a user's data repository granted to a medical service provider is by way of a code populated by the health data module, by biometrics, or a by a chip card.

5. The system of claim 1, wherein the platform is configured to send an alert to a pre-selected medical service provider upon the admission of the user to a hospital.

6. The system of claim 5, wherein the health data module is further configured to capture data created during the user's admission to the hospital including hospital admission notes, medications provided, tests and laboratory results, and imaging.

7. The system of claim 1, wherein the communication module receives input from the user regarding the user's current health status and connects the user with a telehealth medical provider to triage the user based on the user's input.

8. The system of claim 1, wherein the service provider module receives a request from a service provider to join the platform and collects the service provider's information to create an account associated with the platform.

9. The system of claim 8, wherein the service provider account in the platform is associated with an online marketplace in the platform for users to view and select services from a plurality of service provider accounts.

10. The system of claim 1, wherein the health data module is further configured to capture scheduling information associated with the user and maintain the scheduling information in a calendaring system stored in the user data repository.

11. The system of claim 1, wherein the platform is configured to send an alert to a pre-selected medical service provider and the user when data received by the lifestyle module indicates risk factors for a plurality of health conditions or disease states associated with the user.

12. The system of claim 1, wherein the health data module receives a user or medical services provider instruction to document and store in the legal document database a do not resuscitate instruction, a power of attorney instruction, or an organ donation instruction.

13. The system of claim 12, wherein access to the legal document database is restricted by the user and only accessible to those provided authorization to access the legal document database associated with the user.

14. The system of claim 1, wherein the health data module, based on the data stored in the user data repository, is configured to provide a hyperlink to a source for education of the user related to a particular item of data stored in the user data repository.

15. The system of claim 1, wherein the health data module, based on the data stored in the user data repository, provides user related trends derived from a plurality of items of data stored in the user data repository and maps the data points over a user selected period of time.
